(19) United States
(12) Patent Application Publication
(10) Pub. No.: US 2009/0254178 A1
(43) Pub. Date: Oct. 8, 2009

(54) Heart Booster Pump With Magnetic Drive

(76) Inventor: Glendal R. Dow, Bedford, TX (US)

Correspondence Address: BRACEWELL & GIULIANI LLP, P.O. BOX 61389, HOUSTON, TX, 77208-1389, US

(21) Appl. No.: 12/417,300

(22) Filed: Apr. 2, 2009

Related U.S. Application Data
(60) Provisional application No. 61/123,090, filed on Apr. 7, 2008.

(51) Int. Cl. A61M 1/12; F04B 17/00

(52) U.S. Cl. 623/3.11; 417/410.1

(57) ABSTRACT

A blood flow pump has a housing with an axis. The pump is mounted concentrically about the axis in the housing, defining a chamber. A pusher plate in the housing is concentric with the axis. The pusher plate is substantially non-rotatable relative to the housing and movable in forward directions along the axis. A pair of driven magnets are mounted to the pusher plate and offset from the axis. A pair of driving magnets are mounted to a support member that is driven by a drive shaft. The driving magnets are offset from the drive shaft so that when rotated, their magnetic fields pass through the magnetic fields of the driven magnets. The magnetic field are arranged to oppose each other, creating a repelling force to cause the pusher plate to push the pump element in a pressure stroke direction.

### FIELD OF THE INVENTION

This invention relates in general to pumps and in particular to a drive mechanism for a heart booster pump.

### BACKGROUND OF THE INVENTION

Mechanical heart pumps are typically external devices temporarily used when a patient is undergoing surgery to repair the heart or to transplant another heart. Mechanical pumps to be implanted are also known but not in extensive use because of the technical problems to be solved. If used for an extended time, the pump ideally should duplicate the human heart. The human heart has a pulse and operates at different blood pressure levels depending upon the type of exertion of the patient. A patient's arteries and veins will naturally expand during exertion, which tends to lower the blood pressure. The patient's arteries and veins will contract while the patient is sedentary, increasing the blood pressure back to an at rest level. Also, while the patient is exercising, in addition to the pulse rate being higher, each stroke of the human heart will pump more blood than while the patient is sedentary.

Rotary heart pumps cannot duplicate a pulse. While reciprocating heart pumps are known, they normally are configured to pump the same volume of blood with each stroke. While workable, changes in blood pressure caused by exertion of the patient are detrimental to the check valves and other components of the pump chamber if the same volume of fluid is pumped with each stroke. Consequently, known reciprocating type heart pumps must be replaced at fairly frequent intervals.

### SUMMARY

The heart pump of this invention has a pump element mounted in a housing to define a chamber. The chamber has inlet and outlet ports for receiving and discharging blood. At least one driven magnet is mounted in the housing in association with the plump element. Movement of the driven magnet in a forward direction results in movement of the pump element from an intake position toward a discharge position. At least one driving magnet is also mounted in the housing for rotation about an axis of the housing. The rotation moves the driving magnet between an aligned or close position and a misaligned or far position relative to the driven magnet. The driven and driving magnets are oriented such that their magnetic forces repel each other as the driving magnet approaches the aligned position. This repelling force causes the driven magnet to move in the forward direction to discharge blood from the chamber.

Preferably, the driven magnet moves linearly along the axis when moving in the forward direction. Preferably the magnets are fixed in orientation to each other so that a maximum repelling force will exist when aligned. The volume of the blood pumped from the chamber will vary in response to the resistance of the vascular system of the patient in which it is implanted. The pump element moves back to an intake position in response to resiliency of the pump element, which is preferably a diaphragm, and blood pressure of the patient. The maximum discharge position and the maximum intake position may vary from stroke to stroke depending upon the whether the patient is sedentary or moving.

The driving magnet may be mounted to a support that is mounted to a drive shaft within the housing. The driving magnet will be offset from the axis of the drive shaft. Rotating the drive shaft rotates the support and thus the driving magnet in a circle. The driven magnet may be mounted to a pusher plate which is mounted in the housing in engagement with the pump element. The pusher plate can move forward and rearward along the axis but is prevented from any significant rotation.

### BRIEF DESCRIPTION OF THE DRAWINGS

FIG. 1 is a front elevational view of a pump constructed in accordance with this invention.

FIG. 2 is a side elevational view of the pump of FIG. 1, and also illustrates by dotted lines a rotary power source.

FIG. 3 is a sectional view of the pump of FIG. 1, shown in an intake position.

FIG. 4 is a sectional view of the pump of FIG. 1, shown in a discharge position.

FIG. 5 is a sectional view of the pump of FIG. 1, taken along the line 5-5 of FIG. 3.

FIG. 6 is a sectional view of the pump of FIG. 1, taken along the line 6-6 of FIG. 3.

### DETAILED DESCRIPTION OF THE INVENTION

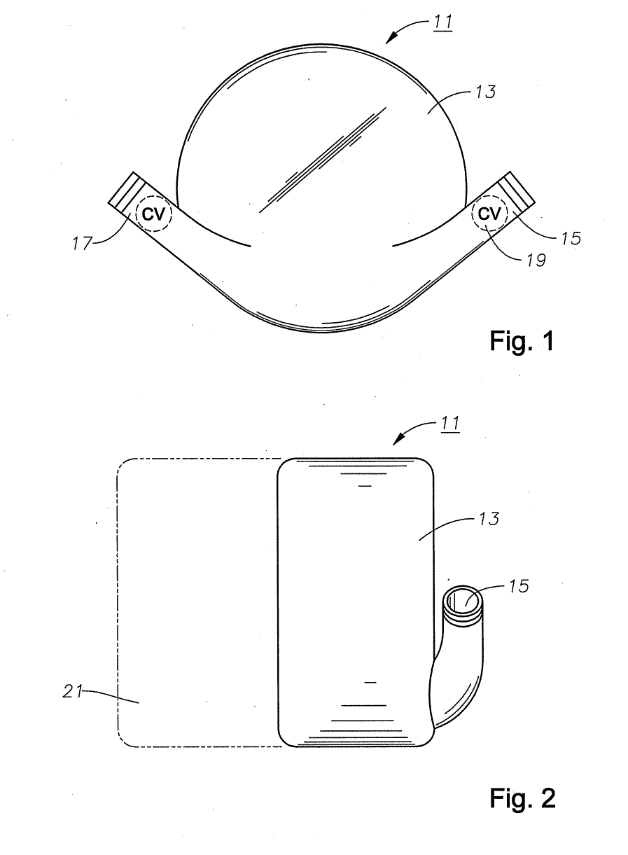

Referring to FIG. 1, pump 11 has a generally cylindrical housing 13, preferably made of a non-magnetic material. Housing 13 has an inlet port 15 and an outlet port 17. Each of the ports 15, 17 has a check valve 19, which is schematically shown.

Referring to FIG. 2, a rotary power source 21 is coupled to housing 13. Power source 21t may also be cylindrical, as shown, and it may have a smaller diameter than housing 13. Power source 21 contains a DC motor with a gear train and batteries for supplying power to the motor (not shown).

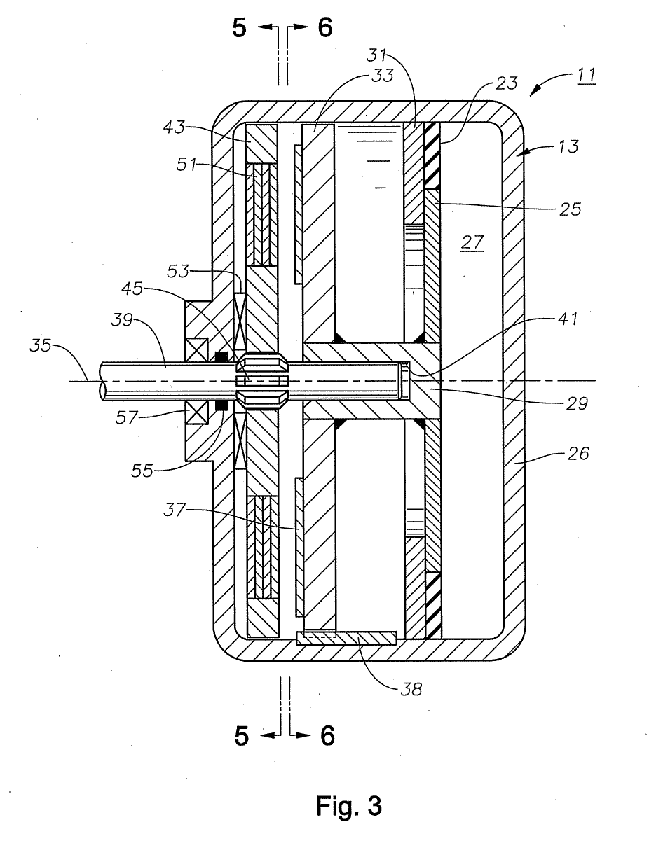

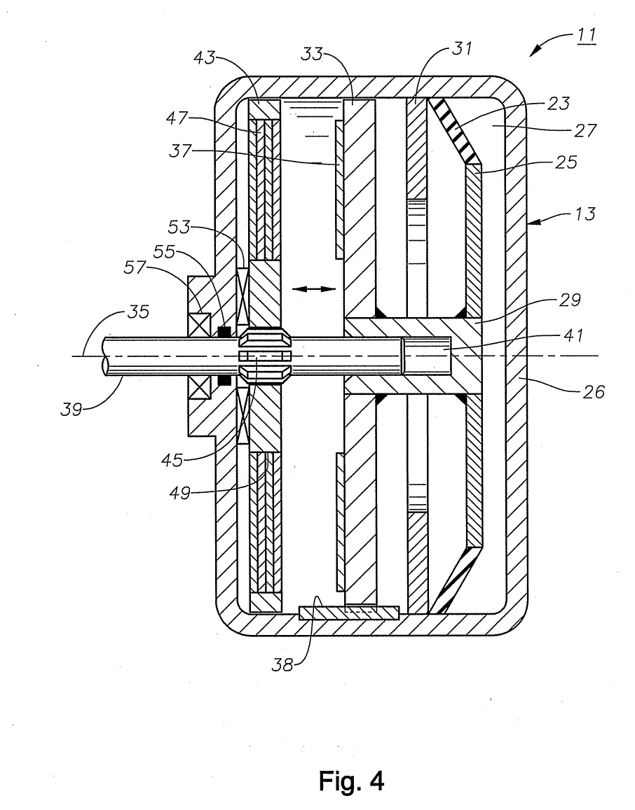

Referring to FIG. 3, a pump element is mounted within housing 13. In this example, the pump element comprises a diaphragm 23 that is of elastomeric material. The elastomeric material may be reinforced with fibers to resist most rotational forces applied to it. Diaphragm 23 has an outer diameter that is bonded to an inner diameter portion of housing 13 to provide a seal and prevent rotation. Diaphragm 23 has an inner diameter bonded to a rigid central plate 25. Diaphragm 23 and plate 25 form a sealed barrier that is spaced from housing forward end wall 26. The space between diaphragm 23 and forward end wall 26 comprises a chamber 27, the volume of which will vary depending upon how close central plate 25 is to end wall 26. As shown in FIG. 4, when in a maximum discharge stroke position, the outer diameter of diaphragm 23 remains fixed in its original axial position, but the inner diameter portion flexes forward as central plate 25 moves toward end wall 26. Inlet port 15 and outlet port 17 are both in fluid communication with chamber 27. While in the intake portion of the pump cycle, shown in FIG. 3, fluid can flow through inlet port 15 into chamber 27. While in the discharge portion of the pump cycle, shown in FIG. 4, blood is forced out outlet port 17 (FIG. 1). Alternate pump elements, such as a piston, could be utilized instead of diaphragm 23 and its central plate 25.

A cylindrical hub 29 is rigidly joined to central plate 25 and extends in a rearward direction, which is the direction to the left. Hub 29 moves in forward and rearward directions along with central plate 25. A stop ring 31 is secured to the inner diameter of housing 13 at a position to limit the maximum intake and discharge strokes. Stop ring 31 is located on the rearward side of diaphragm 23.

In this example, a pusher plate 33 is rigidly mounted to hub 29 a selected distance rearward from central plate 25. Pusher plate 33 is a circular plate with an outer diameter that may be closely spaced to the inner diameter of the cylindrical portion of housing 13. Pusher plate 33 will move in unison with central plate 25 in forward and rearward directions along an axis 35. FIG. 4 shows pusher plate in a position near stop ring 31. In the maximum discharge stroke position, pusher plate 33 may abut stop ring 31, and in the maximum intake stroke position, central plate 25 abuts stop ring 31.

As shown in FIG. 6, pusher plate 33 has on its rearward side a pair of driven magnets 37. Each driven magnet 37 comprises a powerful rare earth magnet, and the strengths of the two driven magnets 37 are preferably about the same. Each has a flat rearward face that faces to the left in the drawing. The rearward faces of the two driven magnets 37 have the same polarity. Driven magnets 37 are preferably bonded to pusher plate 33, such as by an adhesive. Pusher plate 33 optionally may be of a ferrous material, in which case magnets 37 would also be magnetically attracted to pusher plate 33. As an alternate to pusher plate 33, driven magnets 37 could be mounted to central plate 25. In the embodiment shown, the center point of each driven magnet 37 is spaced 180° rotationally from the other. Alternately, the center points could be spaced apart at amounts other than 180° as will be explained subsequently.

Referring again to FIG. 3, pusher plate 33 is preferably restrained against any significant rotation relative to housing 13. The bonding of diaphragm 23 to the inner wall of housing 13 will prevent significant rotation of pusher plate 33, although an incremental amount of rotation is possible due to twisting of diaphragm 23 in response to a rotating magnetic field, described subsequently. In addition to the bonding of diaphragm 23, other devices optionally may be employed to resist rotation of pusher plate 33. In one embodiment, a key 38 is mounted to the inner wall surface of housing 13 and in engagement with a slot in pusher plate 33. The slot slides over stationary key 38 as pusher plate 33 moves axially forward and rearward. Alternately, for example, a pin with a roller could be molded to pusher plate 33 and positioned to roll within slot formed in housing 13. Another alternate would be utilizing opposing magnets that are located at the periphery of pusher plate 33 and the inner diameter of housing 13.

A rotary driven drive shaft 39 extends into housing 13. Drive shaft 39 is driven by power source 21 (FIG. 2) and is preferably located on axis 35. It may have a forward end received within a central cavity 41 formed in hub 29 to maintain hub 29 on axis 35. If so, hub 29 is free to slide axially relative to drive shaft 39 as can be seen by comparing FIGS. 3 and 4.

A support member or plate 43 is mounted to drive shaft 39 for rotation therewith. Support plate 43 is a circular plate similar in outer diameter and thickness to pusher plate 33 in this example. Drive shaft 39 has a splined section 45 or the like for rotating support plate 43. Support plate 43 and drive shaft 39 are restrained against any axial movement relative to housing 13.

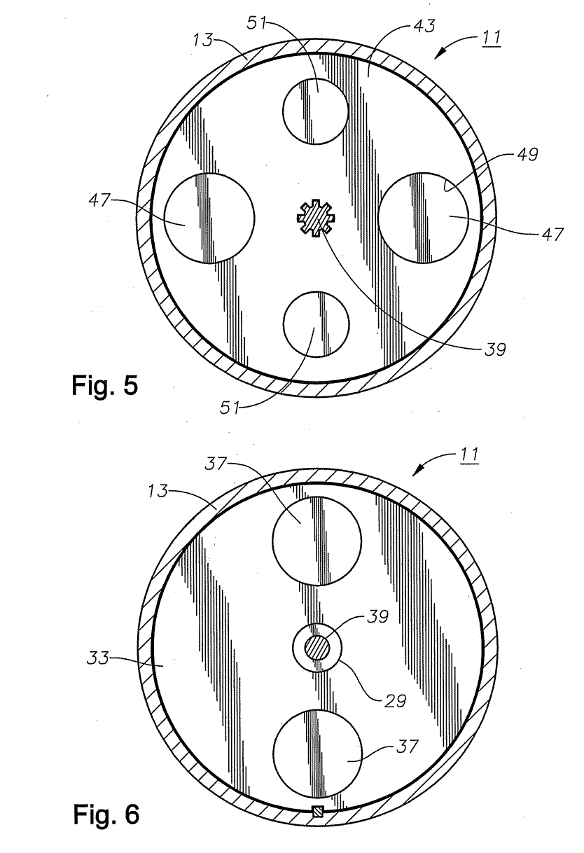

Referring to FIG. 5, at least one and preferably two rare earth driving magnets 47 are mounted to support plate 43. In this example, driving magnets 47 are each located within a hole or cavity 49 formed in support plate 43. Each cavity 49 has a shoulder (not shown) on its rearward side that prevents driving magnets 47 from sliding out the rearward opening of cavity 49. Alternately, cavity 49 could have a closed bottom. In the example shown, each driving magnet 47 is made up of several magnets in the shape of circular disks stacked together within cavity 49; alternately, each driving magnet 47 could be a single element. Each driving magnet has a flat forward face that faces in the forward direction and is parallel to support plate 43. The forward face may protrude from support plate 43 or it may be flush or recessed from support plate 43. In this example, each driving magnet 47 is a circular and has about the same diameter as one of the driven magnets 37, but they can be different. Driven magnets 37 and driving magnets 47 are preferably positioned so that their center points are the same radius from axis 35, but this is not critical. Also, in the preferred embodiment, driving magnets 47 are positioned so that their center points are 180° from each other as shown in FIG. 5, but that can also be varied.

The magnetic field forces of driving magnets 47 are similar to each other and optionally stronger than the forces of the magnetic fields of driven magnets 37, but the forces could be equal or be reversed in strengths. Each driving magnet 47 and driven magnet 37 has a north and south pole, and the forward faces of driving magnets 47 are of the same polarity as the rearward faces of driven magnets 37. Driven magnets 37 and driving magnets 47 are thus oriented so that they will exert repelling forces against each other as they near each other. The repelling force will be maximum at their closest proximity, which is when the center point of one driving magnet 47 aligns with the center point of one driven magnet 37. Driving magnets 47 do not physically touch driven magnets 37 as driving magnets 47 are rotated. Preferably, driving magnets 47 simultaneously align with the driven magnets 37. The combined repelling force is sufficient to change the direction of movement of diaphragm 23 and push diaphragm 23 toward the maximum discharge position. As driving magnets 47 rotate past driven magnets 37, the repelling forces decrease and an attracting force will immediately commence. To avoid overly rapid movement of pusher plate 33 back toward the maximum intake position, dampener magnets 51 are employed. This along with varying filling forces allows for variable filling volumes of chamber 27.

As shown in FIG. 3, dampener magnets 51 are similar to driving magnets 47 in that they are rare earth magnets. There are two dampener magnets 51 in this embodiment, and each is spaced equidistant between the two driving magnets 47, but they do not have to be spaced this way. Dampener magnets 51 are also mounted within a cavity 49, or they may be otherwise bonded to support plate 43. Each dampener magnet 51 could be made up of several thin magnetic disks or each could be a single member of various sizes and shapes and/or positions. Dampener magnets 51 are also oriented with the polarity of their magnetic fields in an opposing relationship to the magnetic fields of driven magnets 37 (FIG. 6). However, dampener magnets 51 have a lesser magnetic strength than driving magnets 47. In this example, the lesser strength is provided by having a smaller diameter than driving magnets 47. The lesser strength acts against the attraction force that occurs immediately after driving magnets 47 pass driven magnets 37. Dampener magnets 51 exert a repelling force that opposes the attraction force and the force due to the resilience of diaphragm 23 (FIG. 3) and the existing pressure within chamber 27. The repelling force of dampener magnets 51 is not sufficient to prevent diaphragm 23 from starting the intake portion of its cycle, but it slows the rate of movement of diaphragm 23 during the intake portion of the cycle.

Referring again to FIG. 3, a thrust bearing 53 is located on the inner surface of the rearward wall of housing 13. Thrust bearing 53 is a flat disc of hard, wear-resistant material. Thrust bearing 53 is contacted by support plate 43 when a reactive force is tending to push support plate 43 rearward or to the left. The reactive force occurs during the discharge portion of the pump cycle.

Drive shaft 39 may have a conventional seal 55 around the hole that it enters in the housing 13. Also, a radial bearing 57 is mounted between housing and drive shaft 39 for rotationally stabilizing drive shaft 39.

In operation, one use for pump 11 is to implant it into a patient with a weak heart. Pump 11 may be located so that inlet 15 is connected to the left ventricle of the patient's heart. The heartbeat of the patient's heart may be controlled by a pacemaker. If a pacemaker isn't employed, the heartbeat may be sensed by control circuitry to power source 21. The control circuitry preferably controls the rotational speed of drive shaft 39 (FIG. 3) to match that of the patient's heart. The control circuitry may vary the rotational speed, but preferably it does not ever completely stop rotation of drive shaft 39. Preferably, the patient's heart is in a discharge stroke or systolic portion of its cycle while pump 11 is in an intake or diastolic portion of its cycle, so that the blood being discharged from the patient's weak heart flows into intake chamber 27 while pusher plate 33 is moving toward or in the maximum intake stroke position. FIG. 3, shows chamber 27 while in the maximum intake volume position. Dampener magnets 51 are momentarily aligned with driven magnets 37.

The continued rotation in the direction indicated by the arrow in FIG. 5 moves dampener magnets 51 past driven magnets and causes driving magnets 47 to rotate 90° from the position shown in FIG. 5 into alignment with driven magnets 37. As driving magnets 47 approach and align with driven magnets 37, the repelling forces increase and begin the systolic portion of its cycle. Pusher plate 33 begins to move forward along axis 35 from the position shown in FIG. 3 toward the position shown in FIG. 4. The length of the stroke will depend on how much resistance there is in the patient's vascular system and other factors. It may be that when driving magnets 47 are perfectly aligned with driven magnets 37, the repelling force is not sufficient to push pusher plate 33 completely to the maximum discharge stroke position. Consequently, the volume of blood discharged from pump 11 may vary per stroke depending on the activity level of the patient.

After driving magnets 47 pass out of alignment with driven magnets 37, dampener magnets 51 will again begin to come into alignment with driven magnets 37. Dampener magnets 51 will start exerting repelling forces once their magnetic fields interact with the opposing magnetic fields of driven magnets 37. The pressure within chamber 27, which is due to the patient's heart, plus the resilience of diaphragm 23 begins the diastolic portion of the cycle, pushing pusher plate 33 back toward the maximum intake position of FIG. 3. Dampener magnets 51 do not have sufficient strength to completely prevent this movement, but will retard the speed of the movement. The forces moving diaphragm 23 toward the maximum intake position may not be adequate to cause it to reach the maximum intake position. As the rotation continues, in another 90°, driving magnets 47 will again begin to exert a repelling force on driven magnets 37. The discharge stroke may begin again before diaphragm 23 reaches the maximum intake stroke position.

As mentioned above, driven magnets 37 may be other than 180° from each other and driving magnets 47 may be other than 180° from each other. For example, measuring from the upper driven magnet 37 in FIG. 6 to the bottom in a counterclockwise direction might be 200°, with the measurement in the counterclockwise direction being 160°. This arrangement would be duplicated with driven magnets 47. The dampener magnets 51 could be spaced equidistant between the driving magnets 47, thus placing them also other than 180° apart from each other. This unequal positioning of magnets 37, 47 and 51 creates two different cycles that alternate with each other. Every other systolic stroke will take longer to complete than the alternating stroke because the amount of rotation would be 200° on one stroke and 160° on the next stroke. An advantage would be to reduce any eddy currents being formed in the blood stream.

Other embodiments include driven, driving and dampener magnets that differ from those shown. For example, all of these magnets could be other than cylindrical discs. A mixture could be employed with the driving magnets 47 being circular and the dampener magnets 51 being some other shape, such as triangular or trapezoidal, or vice-versa. The driving magnets 47 and dampener magnets 51 could be annular or circular rings or the driving magnets 47 and dampener magnets 51 can be any combination of varying sizes and/or shapes so as to vary the characteristics of the forces generated.

While the invention has been shown in connection only showing one of its forms, it should be apparent to those skilled in the art that it is not so limited but is susceptible various changes without departing from the scope of the invention.

1. A blood flow pump, comprising:
a housing;
a pump element mounted in the housing, defining a chamber;
inlet and outlet ports in the chamber for receiving and discharging blood;
at least one driven magnet in the housing and cooperatively associated with the pump element such that movement of the driven magnet in a forward direction results in movement of the pump element from an intake position toward a discharge position; and
at least one driving magnet in the housing, the driving magnet being rotatable about an axis, the rotation moving the driving magnet between an aligned position and a misaligned position relative to the driven magnet, the driven and driving magnets being oriented such that their magnetic forces repel each other when in the aligned position, causing the driven magnet to move in the forward direction when the driving magnet moves to the aligned position.

2. The pump according to claim 1, wherein the driven magnet moves linearly along the axis while moving in the forward direction.

3. The pump according to claim 1, wherein:
the driven magnet has north and south poles, one of which faces forward and the other rearward; and
the driving magnet has north and south poles that face in opposite directions to the poles of the driven magnet.

4. The pump according to claim 1, wherein:
each of the magnets has north and south poles that are fixed in the same directions regardless of the positions of the pump element and the driving magnet.

5. The pump according to claim 1, wherein the volume of blood pumped from the chamber varies in response to the resistance to the blood being pumped from the chamber.

6. The pump according to claim 1, further comprising:
a rotatably driven drive shaft extending into the housing along the axis;
a driving magnet support mounted to the drive shaft within the housing for rotation therewith; and wherein
the driving magnet is mounted to the driving magnet support offset from the axis.

7. The pump according to claim 1, further comprising:
a pusher plate mounted in the housing for forward and rearward movement along the axis and prevented from any significant rotation about the axis; and wherein
the driven magnet is mounted to pusher plate.

8. The pump according to claim 1, wherein each of the magnets is offset from the axis.

9. The pump according to claim 1, wherein the magnets comprise circular disks.

10. A blood flow pump, comprising:
a housing having an axis;
a pump element mounted concentrically about the axis in the housing, defining a chamber;
inlet and outlet ports in the housing in communication with the chamber for receiving and discharging blood from the chamber;
a pusher plate in the housing concentric with the axis, the pusher plate being substantially nonrotatable relative to the housing and movable in discharge stroke and intake stroke directions along the axis, the pusher plate being cooperatively engaged with the pump element for pushing the pump element in the discharge stroke direction to push blood from the chamber through the outlet port;
a pair of driven magnets mounted to the pusher plate for movement therewith, each of the driven magnets being offset from the axis and having a magnetic field that is of the same polarity and faces rearward;
a rotatably driven drive shaft extending into the housing along the axis;
a support member mounted concentrically to the drive shaft within the housing for rotation therewith; and
a pair of driving magnets mounted to the support member for rotation therewith, each of the driving magnets being offset from the drive shaft and having a magnetic field facing forward that has a polarity the same as the rearward facing magnetic fields of the driven magnets, the support member being positioned such that rotation of the drive shaft causes the magnetic field of each driving magnet to rotate through the magnetic field of each driven magnet to exert repelling forces.

11. The pump according to claim 10, wherein centerpoints of the driven magnets are 180 degrees apart from each other relative to the axis.

12. The pump according to claim 10, further comprising:
a pair of dampener magnets mounted to the support member, each of the dampener magnets being offset from the drive shaft and having a magnetic field facing forward that has a polarity the same as but a lesser strength than the magnetic fields of the driven magnets and/or the driving magnets.

13. The pump according to claim 12, wherein the pump element moves in the intake stroke direction in response to a return force due to resiliency of the pump element and pressure of blood entering the intake, and the dampener magnets exert a dampening force opposed to the return force to slow a rate of movement of the pump element in the intake stroke direction.

14. The pump according to claim 12: wherein:
centerpoints of the driven magnets are a selected rotational distance part from each other relative to the axis; and
centerpoints of the dampener magnets are spaced the same rotational distance apart from each other relative to the axis.

15. The pump according to claim 12, wherein:
the driven and driving magnets comprise circular disks.

16. The pump according to claim 12, wherein:
the pump element comprises an annular elastomeric ring having an inner diameter bonded to a rigid hub; and
the pusher plate is attached to the hub for movement therewith.

17. A method of pumping blood, comprising:
providing a housing containing a pump element defining a chamber, inlet and outlet ports in the chamber, and at least one driven magnet and at least one driving magnet;
rotating the driving magnet in a circle so that a magnetic field of the driving magnet passes into and out of a magnetic field of the driven magnet, causing a repelling force to occur each time the magnetic field of the driving magnet passes through the magnetic field of the driven magnet; and
with the repelling force, changing a direction of movement of the pump element from an intake stroke direction, which allows blood flow into the chamber, to a discharge stroke direction, which pushes blood from the chamber.

18. The method according to claim 17, farther comprising:
allowing the pump element to move in the intake stroke direction when the magnetic field of the driving magnet is not within the magnetic field of the driven magnet; and
dampening a rate at which the pump element moves in the intake stroke direction.

19. The method according to claim 18, wherein dampening the rate comprises:
rotating a magnetic field of a dampener magnet through the magnetic field of the driven magnet and exerting a repelling force in response thereto, the magnetic field of the dampener magnet being of less strength than the magnetic field of the driving magnet.
